(19) United States
(12) Patent Application Publication
(10) Pub. No.: US 2025/0020627 A1
(43) Pub. Date: Jan. 16, 2025

(54) AN INSPECTION TOOL FOR INSPECTING A CONCRETE SURFACE

(71) Applicant: HUSQVARNA AB, Huskvarna (SE)

(72) Inventors: Andreas Jonsson, Hallsberg (SE); Jacob Larsson, Molndal (SE); Edvin ALFREDSSON, Goteborg (SE); Bjorn Hagstrom, Huskvarna (SE); Hampus Wallstrom, Huskvarna (SE)

(21) Appl. No.: 18/713,629

(22) PCT Filed: Jun. 16, 2022
(86) PCT No.: PCT/SE2022/050593

§ 371 Date: May 24, 2024

Related U.S. Application Data
(63) Continuation-in-part of application No. PCT/SE2021/051278, filed on Dec. 17, 2021.

(30) Foreign Application Priority Data
Nov. 25, 2021 (SE) 2151434-4

(51) Int. Cl. G01N 33/38; G01B 11/25; G01B 11/30; G01N 21/15; G01N 21/88; G06T 7/00

(52) U.S. Cl. CPC G01N 33/383; G01B 11/2527; G01B 11/303; G01N 21/8806; G06T 7/0004; G01N 21/15; G01N 2021/151; G01N 2021/8829; G06T 2207/10012; G06T 2207/10028; G06T 2207/30132

(57) ABSTRACT

An inspection tool (100) for inspection of a concrete surface (180), the tool comprising a vision-based sensor (110) arranged to be directed at a section of the concrete surface (180), guiding means (120) for allowing an operator (170) to move the vision-based sensor (110) over the concrete surface (180), a trigger (130) arranged to receive a command from the operator (170), wherein the vision-based sensor (110) is arranged to capture at least one image of the concrete surface in response to the command, a control system arranged to analyze the at least one image of the concrete surface in terms of a surface quality of the section of the concrete surface (180), and a display unit (140) arranged to present a result of the surface quality analysis to the operator (170).

### TECHNICAL FIELD

The present disclosure relates to concrete surface processing and in particular to inspection of processed concrete surfaces in order to determine a current state and/or a quality of the concrete surface.

### BACKGROUND

Concrete surfaces are commonly used for flooring in both domestic and industrial facilities. The sizes of concrete surface floors range from a few square meters for a domestic garage floor to thousands of square meters in larger industrial facilities. Concrete surfaces offer a cost efficient and durable flooring alternative and have therefore gained popularity over recent years.

Concrete surface preparation is performed in steps. After the concrete is poured, the surface is first troweled and then grinded flat after the surface has reached a sufficient level of maturity. A matured concrete surface can then be grinded and polished to a glossy finish if desired. Grinding and polishing of a concrete surface is performed using a sequence of tools with finer and finer grit. It is important that the change to a finer grit tool is not performed prematurely, since then the finer grit tool will not be able to remove the scratches in the surface in a reasonable amount of time. It is also important that a given grit is not used for too long, since this is inefficient from a production time perspective and also leads to an unnecessary consumption of grinding tools.

An operator normally determines when to change tools, and when the grinding process is finished, based on ocular inspection of the concrete surface and from general experience. However, such experience takes time to acquire, and experienced concrete surface processing operators are sometimes hard to find.

### SUMMARY

It is an object of the present disclosure to provide concrete surface processing equipment which allow more efficient processing of concrete surfaces.

This object is obtained by an inspection tool for inspection of a concrete surface. The tool comprises a vision-based sensor arranged to be directed at a section of the concrete surface, guiding means for allowing an operator to move the vision-based sensor over the concrete surface, and a trigger arranged to receive a command from the operator, wherein the vision-based sensor is arranged to capture at least one image of the concrete surface in response to actuation of the trigger. The inspection tool also comprises a control system arranged to analyze the at least one image of the concrete surface in terms of a surface quality of the section of the concrete surface, and a data interface and/or a display unit arranged to output and/or to present a result of the surface quality analysis to the operator.

The guiding means allow the operator to deploy the vision-based sensor at desired locations over the concrete surface. At each location, the surface quality can be determined in an accurate manner using the vision-based sensor, conveniently controlled by the trigger. Thus, even relatively large concrete surfaces can be inspected in an efficient manner. The surface quality analysis may for instance comprise analysis of the presence of scratch marks, cracks in the surface, and/or a level of surface gloss. The surface quality analysis may also comprise determination of a suitable grinding tool grit for processing the surface. Various other example concrete surface analysis components will be discussed below.

A light shield is preferably configured to enclose the vision-based sensor and the section of the concrete surface, in order to shield the vision-based sensor from ambient light. This light shield increases the performance of the vision-based sensor, especially in environments with strong ambient light. The light shield also improves the result of performing shape-from-shadow (SFS) techniques and also structured light techniques.

The inspection tool optionally comprises a height detection system arranged to detect a height of the inspection tool relative to a reference height. The height detection system comprises an array of photodiodes, which can be used to establish the height in relation to a reference plane generated by a rotary laser transmitter in a convenient manner and cost efficient manner. The inspection tool may also comprise a surface cleaning arrangement arranged to remove dust and other unwanted material from the surface prior to capture of the at least one image of the concrete surface by the vision-based sensor. The surface cleaning arrangement ensures that excessive amounts of dust is not present on the surface, where it may affect the accuracy of the vision-based sensor, which is an advantage. A lens cleaning arrangement arranged to remove dust from a lens of the vision-based sensor prior to capture of the at least one image of the concrete surface may also be comprised in the inspection tool, in order to keep the vision-based sensor clean and high performing for extended periods of time in environments with dust that could otherwise negatively affect the performance of the vision-based sensor. According to an example, a pressurized gas system for dispensing pressurized gas, such as air or carbon dioxide, can be used to blow gas at high speed into an interior of the 3D camera sensor and/or onto the concrete surface and/or onto a lens of the vision-based sensor.

According to some aspects, the inspection tool also comprises a positioning system arranged to determine the position the inspection tool on the concrete surface. The positioning system allows an association between captured data and location on the surface, which is an advantage. The positioning system, when coupled with the height detection system, also enables generation of a topology map which can be useful when determining concrete surface quality. The positioning system may for instance be based on determination of an angle of departure of a laser beam emitted by a laser transmitter arranged to be supported at a pre-determined distance above a base plane of the concrete surface. This way standard rotary laser transmitters, which are readily available on most construction sites, can be used to position the inspection tool with high accuracy.

The inspection tool optionally also comprises a durometer and/or a device arranged to form a scratch in the concrete surface. The durometer and/or the device is arranged to determine a surface hardness level of the concrete surface. This surface hardness may form an important part of a concrete surface state report, indicating for instance if a given concrete surface processing step, such as troweling or floor grinding, may commence or if the surface is still too immature for the next processing step.

The guiding means may simply be constituted by a handle arranged attached to the vision-based sensor at a distal end of the handle. However, a trolley supporting the vision-based sensor on a bottom part of the trolley can also be used, or a sled arranged to be slidably supported on the concrete surface.

It is furthermore appreciated that the guiding means may comprise a robot, such as a remote controlled robot or an autonomously operated robot. The inspection tool may also be integrated in some other concrete surface processing equipment, such as a floor grinder, a power trowel, or a dust extractor.

The vision-based sensor optionally comprises a 3D camera sensor arranged closer than about 30 cm from the concrete surface, and preferably closer than 20 cm from the concrete surface. This location close to the concrete surface allows the camera to capture the surface in high resolution, showing exceptionally fine detail.

The 3D camera sensor may also comprise one or more light sources which are spatially separated from one or more image sensors, allowing the vision-based sensor to perform an SFS analysis of the concrete surface.

The 3D camera sensor optionally also comprises a plurality of image sensors arranged for stereoscopic vision, allowing the surface topology to be determined with high precision. This stereoscopic vision function has been found to work well in combination with SFS. According to some aspects, the control system is arranged to generate a plurality of 3D representations of the section of the concrete surface by a plurality of image sensors and the control system is arranged to perform a stereoscopic procedure to determine a 3D representation of the concrete surface comprising depth information. This further improves the resolution of the surface image, especially when it comes to small differences in height. To improve resolution even more, the control system can be arranged to perform a plurality of SFS processes and corresponding stereoscopic process for each elevation angle out of a predetermined plurality of elevation angles.

The vision-based sensor optionally also comprises a structured light image sensor. This technique has been found to give good results in determining a surface structure of a concrete surface at fine detail. A projector component of the structured light image sensor is advantageously arranged to operate in a defocused mode of operation. This reduces the resolution requirements on the projector, which is an advantage.

The vision-based sensor advantageously comprises a light source such as one or more light emitting diodes (LED) and/or a projector device arranged to project an image onto the concrete surface, and at least one detector arranged to capture an image of the surface. The light source and the detector are preferably spatially separated from each other. This allows the surface to be better inspected, e.g., in terms of cracks and unevenness, since the concrete surface is illuminated from one angle and observed from another angle.

The inspection tool may also comprise an analog or electronic spirit level arranged to indicate an angle of the inspection tool relative to a vertical reference axis. The spirit level provides information about the surface inclination, which may be valuable, e.g., in order to determine if the concrete surface is sloping or not at some location. The spirit level can also be used to make sure that the tool is positioned correctly, i.e., correctly levelled, before the vision-based sensor is triggered by use of the trigger.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated. Further features of, and advantages with, the present invention will become apparent when studying the appended claims and the following description. The skilled person realizes that different features of the present invention may be combined to create embodiments other than those described in the following, without departing from the scope of the present invention.

### BRIEF DESCRIPTION OF THE DRAWINGS

The present disclosure will now be described in more detail with reference to the appended drawings, where

FIG. 1 illustrates a concrete surface inspection tool in use;

FIG. 2 schematically illustrates a concrete surface inspection tool;

FIG. 3 schematically illustrates an image sensor and a light source;

FIGS. 4-6 illustrate an example vision-based sensor;

FIG. 7 is a flow chart illustrating a method for surface inspection;

FIGS. 8A-B illustrate example concrete surface inspection tools;

FIGS. 9A-B illustrate example concrete surface inspection tools;

FIGS. 10-12 illustrate a vision-based sensor based on structured light;

FIG. 13 illustrates an example 2D concrete surface image;

FIG. 14 illustrates an example 3D concrete surface image; and

FIG. 15 illustrates an example inspection tool positioning system;

FIG. 16 shows an example surface quality inspection report on a display;

FIG. 17 is a flow chart of a method for inspecting a concrete surface;

FIG. 18 schematically illustrates a control system;

FIG. 19 shows a computer program product;

### DETAILED DESCRIPTION

The invention will now be described more fully hereinafter with reference to the accompanying drawings, in which certain aspects of the invention are shown. This invention may, however, be embodied in many different forms and should not be construed as limited to the embodiments and aspects set forth herein; rather, these embodiments are provided by way of example so that this disclosure will be thorough and complete, and will fully convey the scope of the invention to those skilled in the art. Like numbers refer to like elements throughout the description.

It is to be understood that the present invention is not limited to the embodiments described herein and illustrated in the drawings; rather, the skilled person will recognize that many changes and modifications may be made within the scope of the appended claims.

Concrete surfaces can be manufactured in a wide variety of textures and appearances, ranging from course unfinished surfaces to polished surfaces with high gloss. Some concrete surfaces are required to be very even, i.e., without bumps and other variations in surface height, while other surfaces are associated with less strict requirements on surface evenness. Some concrete surfaces may be required to exhibit a certain look, such as a certain level of gloss, which requires a given amount of material to be removed from the surface, but without strict requirements on the surface having a uniform level. Concrete surface processing may comprise grinding with a purpose to produce an even surface, or alternatively with the purpose to grind away a given amount of material over the surface to produce a certain look.

As mentioned above, it is often desired to analyze the concrete surface in order to determine a current surface quality, e.g., in terms of the amount and size distribution of scratch marks, surface level evenness, gloss and texture. The result of the analysis can be used to determine if a required surface quality has been achieved, i.e., if a target specification has been met, or if more work is needed. The analysis can also be used to determine a suitable tool grit for grinding or polishing a surface, i.e., if the concrete surface has sufficiently small scratch marks to move on to the next level of tool grit, or if further processing is necessary before change of tools. This type of concrete surface analysis has traditionally been performed manually by visual or tactile inspection. However, this requires a significant amount of experience, and is obviously not a very consistent method.

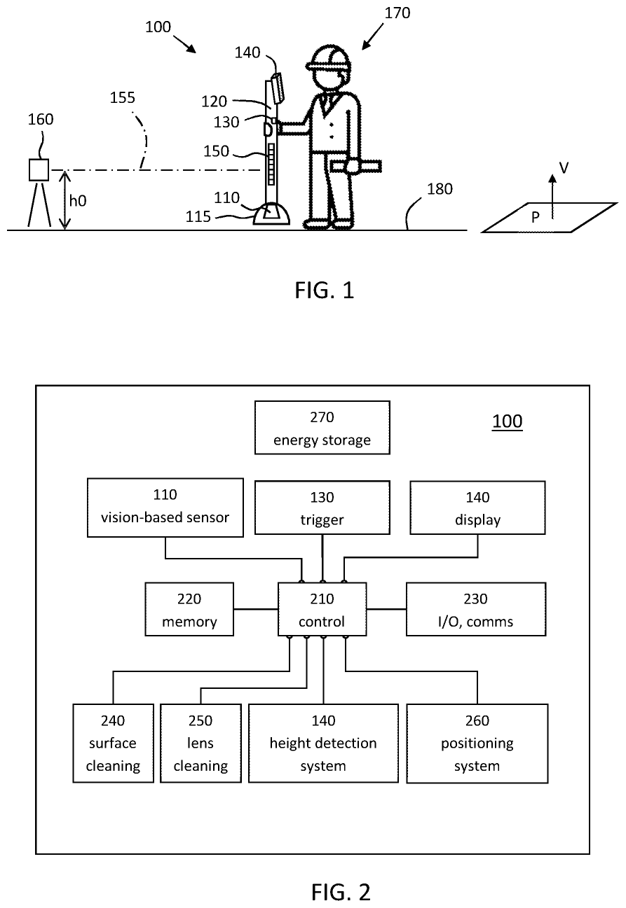

FIG. 1 illustrates an inspection tool 100 for manual inspection of a concrete surface 180 by an operator 170. This tool 100 allows for consistent and automated analysis of concrete surface quality, which means that inexperienced operators can also use the tool while still providing accurate and consistent analysis results. The tool can, for instance, be used to determine a current surface quality, and thus to determine if a surface quality specification has been met or if further processing of the concrete surface is necessary to meet the specification. The inspection tool can also be used to determine if the processing of the concrete surface using a given grit has been completed, i.e., if processing by a finer grit tool can start, or if more processing by the same grit tool is required.

The tool 100 comprises a vision-based sensor 110 arranged to be directed at a section of the concrete surface 180 to be examined. Examples of suitable vision-based sensors will be discussed in detail below. The vision-based sensor is preferably but not necessarily enclosed by a light shield 115 that protects the sensor from ambient light which could otherwise have a negative effect on the image-data obtained from the vision-based sensor 110. The light shield 115 can, for instance, comprise some form of skirt or housing which encloses the image-based sensor 110 and engages the concrete surface so as to prevent light from entering into the interior of the light shield 115. The rim of the light shield 115 may, e.g., be made of a resilient material such as rubber, or comprise a dense brush which makes sealing contact with the concrete surface. The light shield 115 does not have to be totally sealing, although the more light that is blocked from entering into the interior of the light shield the better it normally is.

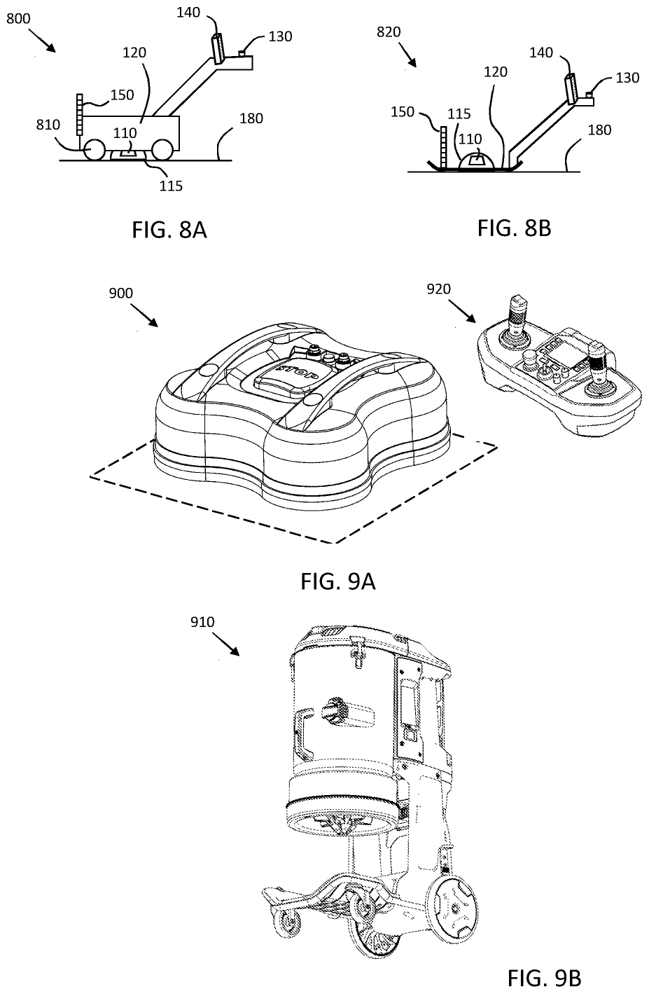

The tool 100 comprises guiding means 120 for allowing an operator 170 to move the vision-based sensor 110 over the concrete surface 180, and to conveniently deploy the analysis tool at a desired concrete surface location. In the example of FIG. 1, this guiding means is essentially a handle, where the vision-based sensor has been mounted at a distal end of the handle. The operator 170 is able to conveniently relocate the vision-based sensor, using the handle, to sample concrete surface quality at desired locations as illustrated in FIG. 1. FIGS. 8A and 8B illustrate alternative guiding means which can be used by the operator to move the vision-based sensor 110 around on the concrete surface 180. FIG. 8A shows a trolley-like design with wheels 810 that support the vision-based sensor on the concrete surface 180. The operator pushes or pulls the trolley around in order to relocate the vision-based sensor 110 and analyze different sections of the concrete surface 180. FIG. 8B shows a sled-like contraption 820 which can be pulled or pushed around on the concrete surface 180 in order to relocate the vision-based sensor to different sections of the concrete surface 180. It is appreciated that many different types of guiding means are possible, and that the term therefore should be construed broadly herein.

In addition to the manual guiding means discussed above, i.e., the trolley, sled-like contraption or handle device, the inspection tool can also be integrated in a robot, such as a remote controlled robot or a robot arranged to move autonomously over the concrete surface 180, whereby the operator can obtain inspection results. The inspection tool may also be integrated in some other concrete surface processing equipment, such as a floor grinder, a power trowel, or a dust extractor. This allows an operator to inspect the surface conveniently during concrete surface processing. In such mountings, the pressurized air system may be applied with advantage to keep the vision-based sensor free from dust. FIG. 9A illustrates a robot 900 which can be adapted to carry the inspection tools discussed herein. The robot may be an autonomously controlled robot configured to inspect a concrete surface in an autonomous manner. The robot 900 can also be controlled via remote control 920. FIG. 9B illustrates a dust extractor 910, which is an example of concrete surface processing equipment where the inspection tool can be integrated. Both the robot and the dust extractor can be fitted with a self-propulsion device, such as an electric motor arranged to propel the machine over the concrete surface, thereby also moving the vision-based sensor between inspection locations on the concrete surface. Note that the operator still operates the inspection device, even if this operation only comprises activating an autonomous robot carrying the vision-based sensor. In case of autonomous operation, said trigger is constituted by the activation of the robot to perform the inspection task.

A trigger 130, such as a pushbutton or touchscreen control, is arranged to receive a command from the operator 170. The operator 170 can use the trigger to start the concrete surface analysis process by the inspection tool 100, 800, 820, 900, 910 in a convenient manner. The trigger can be located close to the operator hand, as illustrated in FIG. 1, or integrated with a light shield around the sensor. When the light shield is pressed against the ground, a pressure switch may trigger the vision-based sensor to capture one or more images of the section of concrete surface. Generally, the vision-based sensor 110 is arranged to capture at least one image of the concrete surface in response to the command by the operator, which image then forms basis for the analysis of the concrete surface. The vision-based sensor 110 may comprise one or more sensor arrangements, such as a high definition digital camera, a shape from shading arrangement, or a sensor arrangement implementing a structured light technique for generating a three-dimensional (3D) reconstruction of a section of the concrete surface 180, i.e., a projector and a camera.

With reference to FIG. 2, the inspection tool 100 also comprises a control system 210 arranged to analyze the at least one image of the concrete surface in terms of a surface quality of the section of the concrete surface 180. The control system may comprise one or more processing circuits, forming part of one or more control units. The analysis of the concrete surface may, e.g., comprise characterization of scratch marks according to depth, detection of cracks in the surface, and/or determination of a level of surface gloss at a given location on the concrete surface 180. The analysis may also comprise a characterization of the section of the concrete surface 180 in terms of evenness and/or comprise determination of a suitable grinding tool grit for continued processing of the surface 180.

The inspection tool 100 preferably comprises an electrical energy storage device 270 arranged to provide electrical power to the inspection tool 100, a data storage device 220 configured to store an amount of data associated with the concrete surface 180 such as image data from the vision-based sensor 110, and also an input/output circuit for data, and/or a wireless communications transceiver 230. Many vision-based sensors generate a substantial amount of data during data capture. Hence, the data storage device 220 may be arranged as a removable data storage device, which the operator can remove from the tool in order to perform further processing of the captured data. A removable data storage device may, e.g., be a removable hard drive, a removable memory, or the like.

A display unit 140 can be arranged to present a result of the surface quality analysis to the operator 170, which may then act in dependence of the report. The display unit 140 may just comprise one or more indicator lights, which is considered to be a rudimentary form of display unit herein, or a more advanced display unit such as a high definition touchscreen device or the like. The operator may, for instance, decide if it is time to change to a finer grit, or if the concrete surface processing operation has resulted in a surface quality according to specification, such that the concrete surface processing operation is finished, based on data communicated to the operator 170 via the display 140. The display unit 140 may not be necessary, e.g., in case the inspection tool instead comprises a data interface 230 arranged to output the result of the surface quality analysis for further analysis elsewhere.

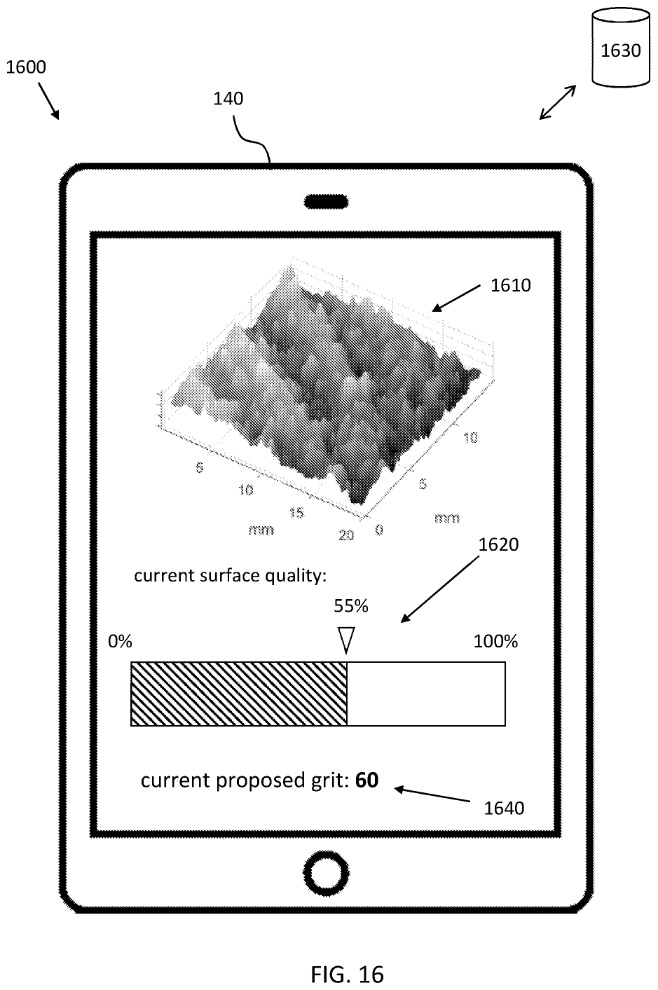

An example concrete surface analysis report 1600 shown on an example display 140 is illustrated in FIG. 16. This example report comprises a visual indication 1610 of the surface condition of the concrete surface 180, which is based on data captured by the vision-based sensor 110. This visual indication allows the operator 170 to determine the surface quality in a convenient manner. The visual indication can also be used to determine a magnitude of scratches and other defects. One or more key metrics can be determined and used to indicate the surface quality. For instance, a height variation metric can be defined as

$${h}_{var} = \frac{1}{N} {\sum }_{i = 1}^{N} {( {h}_{i} - \overline{h} )}^{2}$$

where N height samples have been taken, and h is the average height of the samples. Other metrics can of course also be defined, such as the maximum depth scratch mark over a section of the concrete surface 180. The operator can visually inspect the 3D reconstruction of the surface, and also compare the surface texture and shape to one or more predefined metrics indicating expected values.

This particular example report 1600 exemplified in FIG. 16 comprises a comparison 1620 of the current surface quality with a target specification, which in this case has been obtained from a remote server 1630. This comparison indicates how much processing that remains until the surface quality of the concrete surface meets the target specifications, i.e., how much work that remains until the concrete surface processing is finished. The report 1600 also comprises a proposal 1640 of a suitable grit to use, which can be determined from the current surface quality, i.e., from a magnitude distribution of the surface unevenness. When the surface quality has improved sufficiently much, the recommendation will be changed to a finer grit tool. This change in recommendation may also be accompanied by a notification signal, such as a buzzer or a flashing light which will notify an operator of the fact. A radio signal can also be transmitted to a personal device of an operator, in order to notify the operator that it may be time to change the tools to another set of tools with finer grit. There are many ways in which the grit proposal can be determined. For example, certain tool grits can be associated with a maximum difference between highest and lowest location on the section of the surface captured by the vision-based sensor, or the variation in height as discussed above. The current determined surface metric can be compared to predetermined ranges, where each range is associated with a given grit. This way the inspection tool can compare the current state of the surface to the ranges and propose a suitable grit to use for further processing. When the surface becomes more smooth from processing, the recommendation will automatically change, since the surface metric will change, and eventually enter into a new range associated with a different (finer) grit.

Referring again to FIG. 1, the inspection tool 100 optionally also comprises a height detection system 150 arranged to detect a height of the inspection tool 100 relative to a reference height. This height detection system 140 is based on a linear array of photodiodes which detects an incoming laser reference beam 155 emitted from a remote laser transmitter 160. Thus, the inspection tool 100 can be a kit of parts that optionally also comprises a laser transmitter 160 arranged to be supported at a pre-determined distance above a base plane of the concrete surface 180 and distanced from the guiding means 120. The laser transmitter 160 may, e.g., be a rotary laser as is common in many construction sites, where they are used to provide a horizontal reference by generating a laser beam which rotates in a plane parallel to some reference surface. The control system 210 of the inspection tool 100 is then connected to at least one linear photo sensor or linear image sensor extending transversally to a base plane of the concrete surface 180, forming part of the height detection system 150. This linear photo sensor is arranged to register an incoming laser beam, such as an incoming laser beam from the rotary laser 160.

A linear photo sensor array is essentially a vertical array of photo sensors. A laser beam hitting a photo sensor in the array will trigger generation of a signal from that photo sensor. The control system 210, being connected to the photo sensor array, can therefore detect the height at which a laser beam strikes the linear photo sensor. A linear photo sensor may also comprise photo sensors arranged in matrix configuration, i.e., in two or more adjacent arrays of photo sensing elements. Such as array is not only able to detect the height at which an incoming laser beam strikes the array but may potentially also detect a tilt of the inspection tool 100 relative to, e.g., the horizontal plane or relative to the base plane.

The height detection system 150, i.e., the combination of the linear photo sensor and the control system 210 arranged to detect the height h enables the inspection tool 100 to determine a surface topology of the concrete surface. By moving the inspection tool 100 around on the concrete surface 180 and measuring the height h at each measurement location, a topology map can be created. The measurement location can either be manually input or obtained from a positioning system. This topology map can then be used to plan or control concrete surface processing in order to arrive at a desired result, such as a flat concrete surface, or a concrete surface which has been grinded down by an equal amount over the surface.

The inspection tool 100 optionally also comprises a surface cleaning arrangement 240 arranged to remove dust from the concrete surface prior to capture of the at least one image of the concrete surface by the vision-based sensor 110. This feature improves the performance of the inspection tool in environments where there is a lot of dust on the concrete surface 180, which may cause the surface to appear more smooth and better polished than it actually is.

A pressurized gas system for dispensing pressurized gas, such as air or carbon dioxide, into the vision-based sensor interior and/or onto the section of concrete surface 180 at which the vision-based sensor is directed has been found to give good results. The pressurized gas system can be triggered by the same trigger 130 as the vision-based system, such that the concrete surface 180 is blown clean from dust and other debris prior to capturing image data of the concrete surface segment. In this case the puff of gas advantageously comes some time before the vision-based sensor captured the data, allowing the dust to be moved away from the section of the concrete surface. The pressurized gas may, e.g., be obtained from a small canister, such as a carbon dioxide canister, often referred to as a CO2 cylinder, commonly used in inflatable life-jackets.

The pressurized gas can of course also be at least in part directed at the image sensor of the vision-based sensor, in order to, e.g., clean the lenses from dust.

Thus, both the concrete surface and the image sensors can be cleaned by compressed gas which forces the dust away from the inspection tool and ensures no dust accumulates on the lens. To summarize according to some aspects, the inspection tool 100 comprises a lens cleaning arrangement 250 arranged to remove dust from a lens of the vision-based sensor 110 prior to capture of the at least one image of the concrete surface by the vision-based sensor 110.

The inspection tool 100 may also comprise a positioning system 260 arranged to position the inspection tool 100 on the concrete surface 180. Various positioning systems can be used with the inspection tool, such as indoor beacon-based positioning systems or the like. In case position data is available, then the concrete surface quality analysis can be associated with a given location on the concrete surface, and the operator can return to the same section of concrete surface to repeat the analysis after the concrete surface has been subject to further processing. A radio-based locationing system can be used, or a laser-beacon based system. The positioning system can also be used in combination with the height detection system 150 to generate a topological map of the concrete surface 180. This topological map may, e.g., be formed by interpolating in-between measurement points on the surface. The topological map can be visualized on the display 140, which may also be arranged to indicate parts of the concrete surface where sufficient topological data is not available, prompting the operator to analyze those parts in more detail, at least when it comes to determining their height profiles.

The inspection tool 100 may furthermore comprise a durometer arranged to determine a surface hardness level of the concrete surface 110. The durometer may comprise a hammer device arranged for determining concrete hardness by determining a rebound energy. Tests using the durometer can also be indicated on the topological map, giving an overview of the current state of the concrete surface. Alternatively or in combination with the durometer, the machine 700 may comprise a device arranged to form a scratch in the concrete surface. The depth of this scratch can then be detected and used to determine a surface hardness level of the concrete surface 110. The depth may be determined using the vision-based sensor 110.

The vision-based sensor 110 may comprise a 3D camera used as surface quality sensor. This camera is preferably located relatively close to the concrete surface, i.e., closer than about 30 cm from the surface, and preferably closer than 20 cm from the concrete surface.

A 3D camera for concrete surface inspection purposes optionally comprises a plurality of spatially separated light sources and one or more image sensors, preferably at least two or three image sensors.

An image sensor is a vision-based sensor that detects and conveys information used to make an image, which can be a color image, a greyscale image, or a representation of infrared radiation from the surface. Two common types of electronic image sensors are the charge-coupled device (CCD) and the active-pixel sensor (CMOS sensor). Both CCD and CMOS sensors are based on metal-oxide-semiconductor (MOS) technology, with CCDs based on MOS capacitors and CMOS sensors based on MOSFET (MOS field-effect transistor) amplifiers. The control system 210 can detect minute scratch marks and other undesired traits in the concrete surface by the output from the 3D camera. The 3D camera sensor is preferably of high resolution, e.g., has a resolution above 10 mega pixels (MP), such as about 13 MP or more.

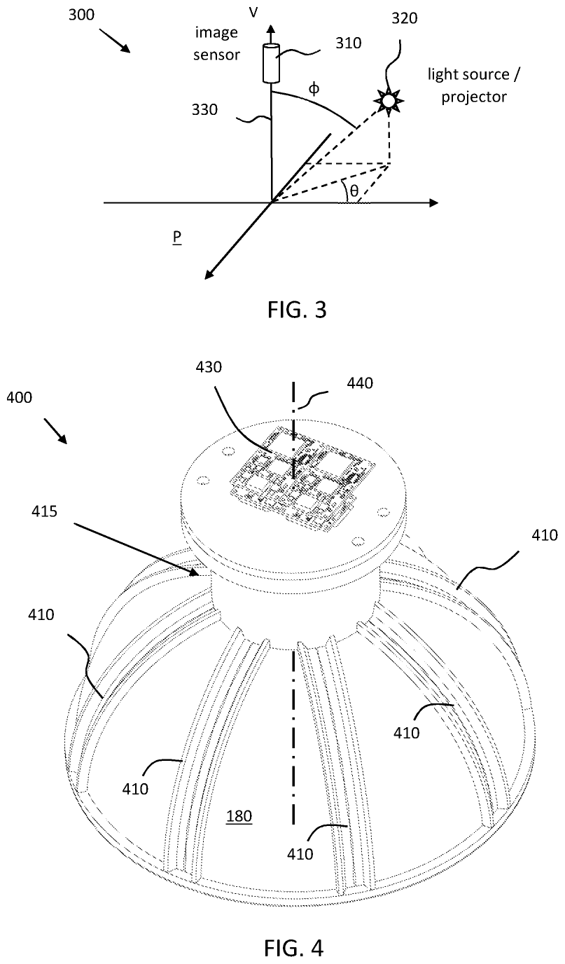

FIG. 3 illustrates one example technique by which a 3D camera arrangement for concrete surface inspection may operate, known as shape-from-shadow (SFS). SFS is a passive noncontact technique for the inference of shape information of a surface or object, such as the topology of the surface, or just the surface heights at different locations and/or the surface normal vectors at different locations. SFS may be used to detect scratches and other imperfections in a processed concrete surface. The SFS technique utilizes the casting of shadows onto a surface to infer the shape of the surface based on how the surface casts shadow when illuminated from different angles. In SFS a static image sensor 310 is normally used to capture several images of a scene as a light source 320 moves over the scene. The light source is preferably a collimated light source, although other types of light sources can also be used. A plurality of fixed light sources can also be used in sequence to illuminate the scene from different directions, i.e., from different angles θ in the surface plane and also different angles q measured relative to a normal vector 330 to the base plane of the concrete surface. With reference to FIG. 3, the angle θ of a light source will henceforth be referred to as a base plane angle, while the angle q will be referred to as an elevation angle.

M. Daum and G. Dudek, provides a description in “Out of the dark: Using shadows to reconstruct 3d surfaces,” published in Computer Vision-ACCV′98, Springer Berlin Heidelberg, 1997, pp. 72-79, isbn: 978-3-540-69669-8.

M. Daum and G. Dudek also discuss SFS in “On 3-d surface reconstruction using shape from shadows,” in Proceedings. 1998 IEEE Computer Society Conference on Computer Vision and Pattern Recognition, 1998, pp. 461-468. doi: 10.2109/CVPR.1998.698646.

Thus, although SFS has not been previously applied to concrete surface inspection in the manner discussed herein, it is a relatively well-known technique and will therefore not be discussed in detail herein.

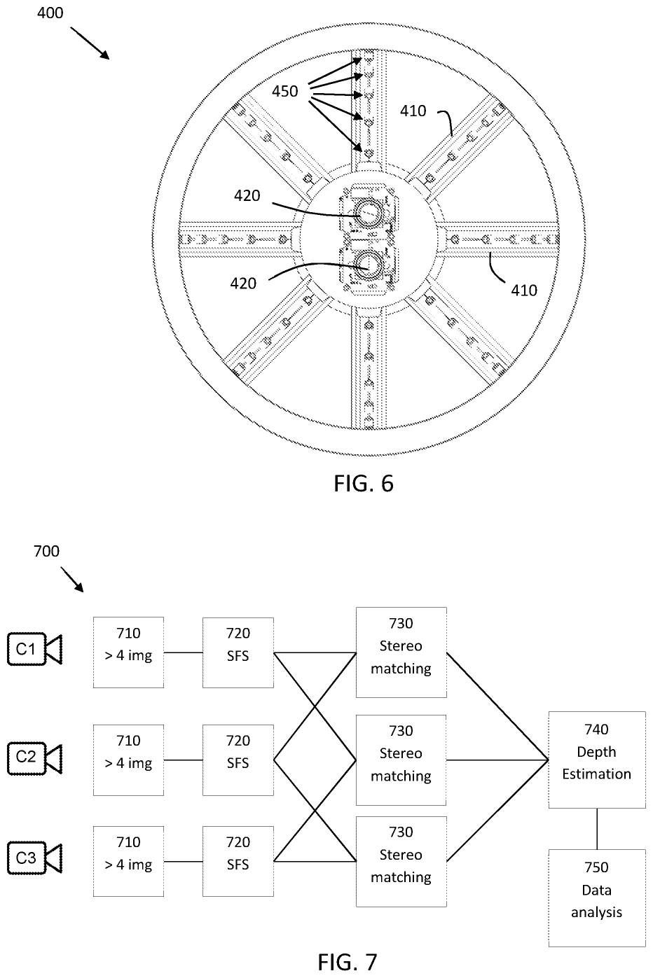

FIGS. 4-6 illustrate an example 3D camera arrangement 400 which comprises a plurality of light sources arranged for SFS operation, or at least a plurality of attachment points configured to hold respective light sources arranged for SFS operation. The light sources are arranged at different base plane angles θ and elevation angles q.

The light sources are preferably collimated LED light sources arranged on arms 410 which extend from a center location or hub 415 intersected by a centrum axis 440 and downward towards the base plane. Processing circuitry 430 and image sensors 420 are arranged in connection to the center location 415. The processing circuitry 430 may be arranged to control both the light sources and the image sensors. The processing circuitry 430 may also be arranged to perform signal processing for surface inspection, although this functionality may also be performed by some other processing resource, perhaps at a remote device, although the amount of data to be transferred to this type of remote processing resource may be prohibitively large.

The arms 410 in the example 400 are of arcuate form and each arm 410 is arranged to carry 6 LED light sources at different elevation angles from about 10 degrees to about 60 degrees. Since there are eight arms in the example 400, the base plane angles are separated by 45 degrees. It has been found that a relatively high elevation angle is advantageous when performing concrete surface inspection. However, it may be even better to use more than one elevation angle in the analysis. The whole image sensor and light source arrangement is preferably shielded from ambient light, e.g., by a light protecting skirt or wall (not shown in FIGS. 4-6).

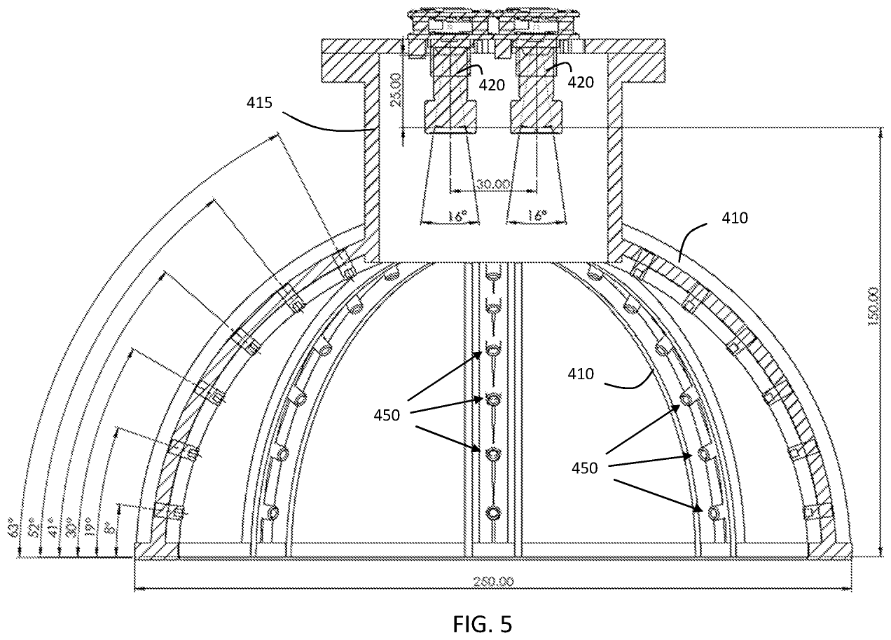

FIG. 5 illustrates the LED light source arrangement in more detail. The light sources 450 are here arranged at elevation angles of 8, 19, 30, 41, 52 and 63 degrees. The light sources are individually controllable from the processing circuitry 430. Thus, it is possible to illuminate a section of the concrete surface from 48 different angles. It has been realized that illumination from different elevation angles may result in different performance depending on the type of defect that is to be analyzed. Deep narrow cracks for instance, are often better seen when illuminated from a large elevation angle, i.e., about 40-60 degrees, while smaller protruding parts on the surface are more clearly seen when illuminated from smaller elevation angles, such as by a light source at an elevation angle of about 10-20 degrees.

It is appreciated that the dome shape in FIGS. 4-6 is just an example. A pyramid shape or some other form of volume can also be used. The main concept being that the light sources are spatially separated and arranged to illuminate the concrete surface from different pre-determined directions. The light protection offered by the light shield 115 is also an important feature which improves the signal to noise ratio in the image data captured by the image sensors 420.

A plurality of image sensors 420 are directed towards the base plane, as shown in FIG. 5. In this case two image sensors are used, although three or more image sensors can also be used. Each image sensor has a field of view of about 16 degrees in this example.

The image sensors are configured to capture a square image of the concrete surface corresponding to about 40 mm by 40 mm, and are arranged in known relation to each other and to the concrete surface. This known spatial relationship enables stereoscopic vision, as will be explained in the following. FIG. 7 is a flow chart 700 illustrating an example method for generating input data to a concrete surface inspection process. The example comprises three image sensors C1, C2, C3 but any plural number of image sensors can be used.

First, for all cameras C1, C2, C3 an SFS procedure 720 is executed based on four or more images captured with different light configurations 710a, 710b, 710c, which results in respective 3D reconstructions of the surface.

Each SFS surface reconstruction is then paired with one other SFS surface reconstruction and fed to a stereo matching module 730, which determines depth over the concrete surface. In traditional stereo vision, two image sensors, displaced horizontally from one another are used to obtain two differing views on a scene, in a manner similar to human binocular vision. By comparing these two images, the relative depth information can be obtained in the form of a disparity map, which encodes the difference in horizontal coordinates of corresponding image points. The values in this disparity map are inversely proportional to the scene depth at the corresponding pixel location.

The output from the different stereo matching processes is then fed to a depth estimation stage 740. The depth estimation stage merges the information obtained from the different stereo matching processes into a final 3D reconstruction of the concrete surface section, which was in view of the image sensors C1, C2, and C3. The data from the depth estimation stage, i.e., an estimated topology of the concrete surface section, is then fed to a data analysis module 750 which formats the data and performs further analysis of the concrete surface section, as discussed herein.

FIG. 8A and FIG. 8B where discussed above. They illustrate alternative guiding means that can be used to deploy the vision-based sensor 110 and the rest of the components of the inspection tool at various locations over the concrete surface. The guiding means 800, 820 can be arranged to carry one or more different types of vision-based sensors 110.

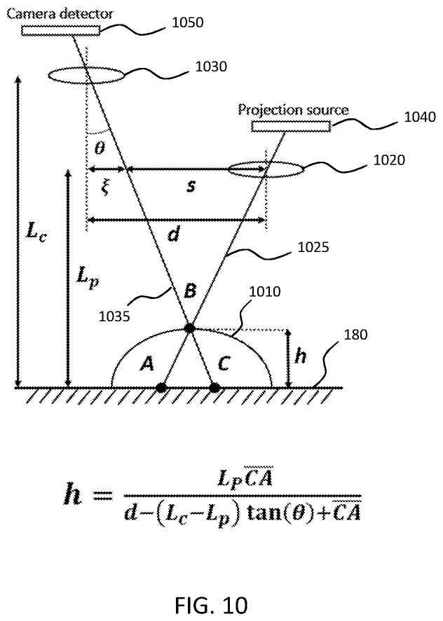

FIGS. 10-12 illustrate another type of vision-based sensor 110. This sensor is based on structured light, and it can be used as a stand-alone sensor or in combination with one or more other types of vision-based sensors. Structured light is a relatively well-known technique for determining the shape of a surface, although it has not been used for concrete surface inspection previously. A known image pattern is projected onto the surface by a projection source and captured by a camera. The distortion of the known pattern is then used to infer the shape of the surface. Tests have indicated that good inspection results can be obtained using the technique, in a cost efficient and robust manner.

Particularly good results have been obtained using a structured light method generally referred to as phase shifting, and more specifically as phase shifting using sinusoidal fringes. This method creates a phase map of the surface using images of a projected sinusoidal fringe pattern that has been phase shifted between images. The phase map can then be used to calculate height using triangulation techniques and basic signal processing. To create the sinusoidal fringes a method called defocused binary pattern (DBP) can be used. DBP creates sinusoidal fringes by projecting stripes and defocusing the projector so that the lines blur and smooth the stripes into a sinusoidal pattern.

FIG. 10 illustrates some basic mathematical relationships that can be used to infer the height of a surface unevenness 1010 at a section of the concrete surface 180, such as a bump or a scratch mark, using a structured light technique. A projection source 1040 emits light which forms an image on the surface. The projected image is then captured by a camera detector 1050, and the control system 210 analyzes the distortion in the image to infer the shape of the surface. Both the projector 1040 and the camera 1050 forms part of the vision-based sensor 110 of the inspection tool 100. In the example of FIG. 10, a triangle A-B-C is formed, where the top corner of the triangle B is at the height of the surface. Point B is also where the projector ray 1025 intersects with the detector ray 1035 on the object surface. Points A and C are where the detector ray 1035 and projector ray 1025 would intersect with the reference surface 180, respectively. Lp and Lc are the distances from the reference surface 180 to the projector lens 1020 and detector lens 1030 respectively, parallel to the optical axis of the lenses. Distance s is the distance between the projector lens and the detector ray normal to the optical axis. & is the distance from the end of s to the detector lens as shown in FIG. 10. Note that if Lp equals Lc then setting & to zero implies that s is also equal to d. To calculate the height h one can use the fact that the two triangles created above and below point B are similar triangles, see, e.g., “White light and x-ray digital speckle photography”. PhD thesis by Per Synnergren, Luleå University of Technology, 2000.

By straight forward mathematical analysis, using said similar triangles, the height h of the surface at the triangle corner B can be determined as

$$h = \frac{{L}_{p} \overline{CA}}{d - ( {L}_{c} - {L}_{p} ) tan ( θ ) + \overline{CA}}$$

where C̅A̅ is the known distance between corners A and C of the assumed triangle.

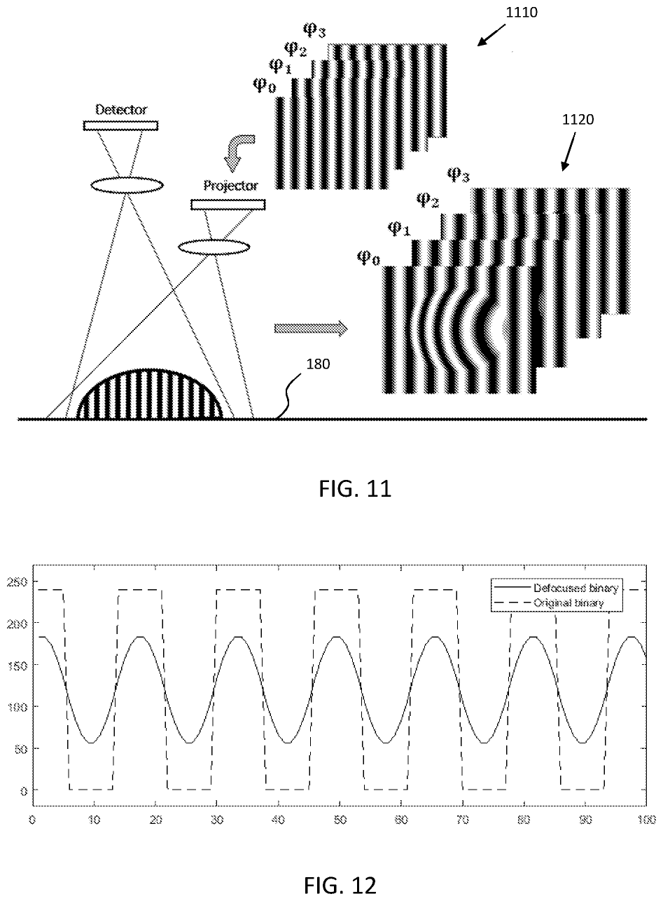

FIG. 11 illustrates projection of a known image pattern onto the concrete surface 180. A series of images, here sinusoidal intensity patterns 1110 of varying phase, are projected onto the surface, and the resulting distorted images are captured by the detector (the camera). The captured images then forms input to a structured light algorithm for determining surface structure.

When projecting sinusoids onto a surface the detected point-wise intensity Iₙ at coordinates (x, y) can be modeled as

$${I}_{n} ( x , y ) = A ( x , y ) + B ( x , y ) cos ( ϕ ( x , y ) - \frac{2 π n}{N} )$$

where n∈[0, N−1] and N is the total number of phase shifts used in the data capture process for each section of the concrete surface to be analyzed. A(x, y) is the background intensity and B (x, y) is a reflection coefficient.

ϕ(x, y)=ϕₒ(x, y)+ϕ_{f}(x, y) is the phase map that contains both the object phase ϕₒ(x, y) and the carrier phase ϕ_{f}(x, y). With the object phase being of interest. The model of Iₙ(x, y) has three unknowns and thus a minimum of three measurements are needed to solve for the phase map @ (x, y). Increasing the number of measurements have benefits such as increased robustness and noise suppression, but the more measurements the longer time the data capture also takes. A common approach is to use four phase shifts, as shown in FIG. 11, where sinusoidal patterns of intensity are projected onto a circular bump, showing both the four projected images 1110 and the detected intensity images 1120 observed from the detector.

Note the fringe deformation between the projected and detected image for φ0, this deformation is caused by the object phase, do.

To tie this object phase ϕₒ(x, y) with the actual height, the distance C̅A̅ in FIG. 10 can be examined in more detail. If a sinusoidal pattern is projected onto the reference surface 180, the distance CA could be expressed as the phase difference times the fringe spacing of the projected images as

$$\overline{CA} = {ϕ}_{C} - {ϕ}_{A} \frac{λ}{2 π} = {\overline{ϕ}}_{CA} \frac{λ}{2 π}$$

where λ is the fringe spacing, ϕ_{c} is the phase value at point C and PA is the phase value at point A. Further if we look at the projection ray in FIG. 10 it shows that the projected intensity, which is constant along this ray will be the same at point B as in point A, leading to ϕ_{B}=ϕ_{A}. The phase difference can then be rewritten as ϕ_{CA}=ϕ_{CB} instead, which allows to use the detected intensity of the object relative the intensity on the reference surface 180, rather than the distance along the reference surface to calculate the height h.

The height is then proportional to the fringe spacing and angle between the detector and projector lens. A higher angle will increase the distance CA and thus increase the resolution. The size of the fringe spacing can be interpreted as a scaling factor, i.e., the larger the fringe spacing the smaller the phase difference will be between point A and C. Desirable for measuring small objects is thus a large angle and a small fringe spacing. Note however that a too large angle will cause shadows from the projector or blind spots from the camera.

The height h for a given point (x, y) on the surface can now be determined as

$$h ( x , y ) = \frac{L {ϕ}_{CB} ( x , y ) \frac{λ}{2 π}}{d - ( {L}_{c} - {L}_{p} ) tan ( θ ) + {ϕ}_{CB} ( x , y ) \frac{λ}{2 π}}$$

which shows that the object phase relative to the phase of the reference surface determines the height h. As mentioned earlier the phase ϕ(x, y) can be extracted from the model of intensity Iₙ(x, y) using multiple measurements of the same surface section by the vision-based sensor 110. In the case of four measurements a complex amplitude containing this phase can be derived as

$$U ( x , y ) = D ( x , y ) exp ( i ( ϕ ( x , y ) ) ) = {I}_{0} ( x , y ) + {iI}_{1} ( x , y ) - {I}_{2} ( x , y ) - {iI}_{3} ( x , y )$$

where D(x, y) is a real amplitude coefficient and ϕ(x, y) is the superposition of the object and carrier phases. It is the detected intensity from the i: th measured phase shift.

The use of a complex amplitude containing the phase rather than solving for the phase directly is that the phase from an object measurement will be compared to the phase of a reference measurement. For the complex amplitude case the phase difference can be extracted as

$$Δϕ ( x , y ) = ∠ {U}_{1} ( x , y ) {{U}_{2} ( x , y )}^{*} = ∠exp ( i ( {ϕ}_{i} ( x , y ) - {ϕ}_{2} ( x , y ) ) )$$

This method for comparing phase maps is more robust around phase jumps rather than just subtracting individual phase maps. Since this is a relatively well-known technique from a general application perspective, no further details will be given herein. Similar techniques, applicable with the inspection tools discussed herein, as discussed by Joaquim Salvi, Sergio Fernandez, Tomislav Pribanic, and Xavier Llado in “A state of the art in structured light patterns for surface profilometry”, Pattern Recognition, 43(8):2666-2680, 2010. See also “Phase Shifting Interferometry, chapter 14, by Horst Schreiber and John H. Bruning, pages 547-666, John Wiley & Sons, Ltd, 2007, and “Phase shifting algorithms for fringe projection profilometry: A review”, by Chao Zuo, Shijie Feng, Lei Huang, Tianyang Tao, Wei Yin, and Qian Chen, Optics and Lasers in Engineering, 109:23-59, 2018.

A problem when wanting to analyze structures at a small scale, on the order of tens of micrometers, is that the projector (the image source) needs to be of quite high resolution. To reduce the requirements on projector resolution, it is possible to use a square pattern, and to defocus this square pattern, This defocusing then effectively results in a more sinusoidal pattern as illustrated in FIG. 12. The defocusing can be implemented as a convolution operation by a Gaussian filter.

The textbook “Time-of-Flight and Structured Light Depth Cameras: Technology and Applications”, by Pietro Zanuttigh, Giulio Marin, Carlo Dal Mutto, Fabio Dominio, Ludovico Minto, and Guido Maria Cortelazzo, Springer, 2016, ISBN-13:978-3319309712, provides an overview of some structured light techniques, of which at least some may be used in the analysis tools discussed herein.

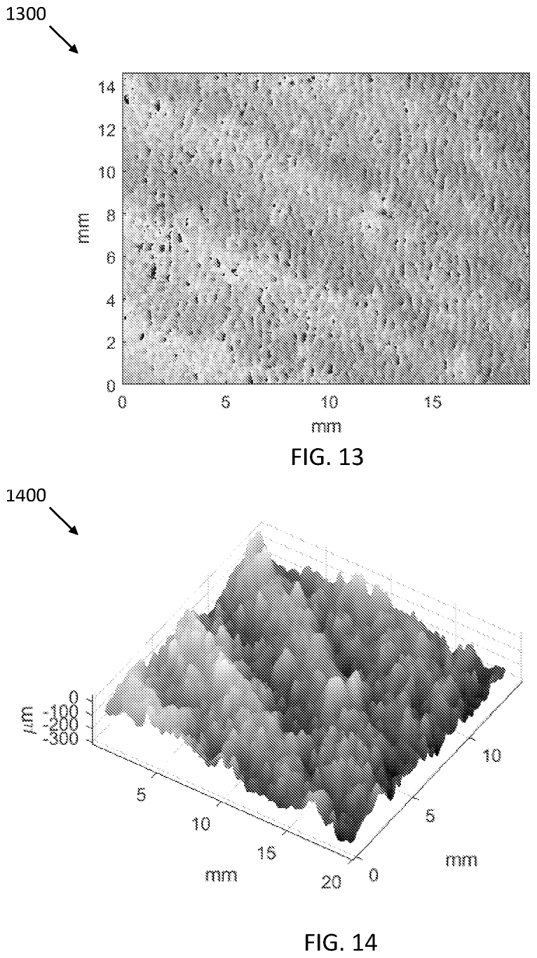

FIG. 13 illustrates an example 2D image 1300 obtained, e.g., using the SFS technique or as a phase map of the projected image of a structured light technique. This type of image can be processed to obtain a representation of the surface in terms of height, i.e., a 3D reconstruction of the surface, as illustrated in the example 1400 of FIG. 14.

The captured image data, and the resulting 3D reconstructions may consume considerable data storage resources. In order to store all the data, the concrete surface processing machine may comprise an on-board data storage device 220 as illustrated in FIG. 2, such as a hard drive or a memory card. The capacity of this memory may be on the order of terabytes or more. The on-board data storage may be arranged to be accessed from the outside via a high-speed data port on the concrete surface processing machine.

The inspection tools 100, 800, 820, 900, 910 discussed herein optionally comprises a positioning system arranged to position the inspection tool on the concrete surface which is being inspected. The positioning system allows inspection data to be associated with a specific position on the surface, and the obtained inspection data can advantageously be indexed by concrete surface position. For instance, if a defect on the surface is discovered, such as a deep scratch or other blemish, then the position of this defect can be logged and later included in an inspection report. The positioning system is of course also important when generating a topology map over the concrete surface, using height data obtained from the height detection system 150.

Various positioning systems are known, which can be used together with the inspection tool. For instance, indoor locationing systems based on beacons are known, as well as camera-based simultaneous locationing and mapping (SLAM) systems. Indoor locationing systems based on ultra-wide band radio signal transmission could be suitable. The global positioning system (GPS) can of course also be used in the inspection tool 100, 800, 820, 900, 910 on locations where such signals are available.

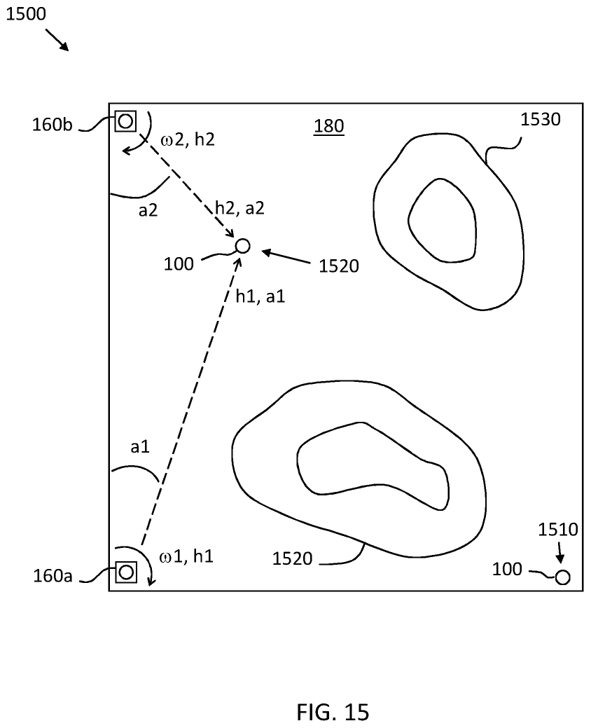

A positioning system 1500 which is particularly suitable for use with the inspection tools discussed herein is illustrated in FIG. 15. Here, at least two rotary laser transmitters 160a, 160b have been deployed at known locations on the surface, preferably in corners of the surface with good visibility from locations over the surface. Each laser emits a rotating laser beam at a respective constant rotation speeds ω1, ω2, and at a respective height h1, h2. The heights are different, allowing each rotary laser to be distinguished at the inspection tool, since the laser beams strike the linear photo sensor comprised in the height detection system 150 at different heights. It is appreciated that the time instant when a laser beam is detected at the height detection system of the inspection tool is related to the angle of departure a1, a2 of the laser beam, thus, if the inspection tool keeps track of when it sees the different laser beams (distinguishable by height of an impinging beam), then it becomes possible to determine the position of the inspection tool on the surface. To summarize, the inspection tools 100, 800, 820, 900, 910 discussed herein optionally comprise a positioning system 260 arranged to position the inspection tool on the concrete surface 180, either in absolute coordinates or relative to some reference point on the surface. The positioning system may be based on a wide variety of different positioning techniques, but a system based on determination of an angle of departure of a laser beam emitted by a laser transmitter arranged to be supported at a pre-determined distance h1, h2 above a base plane of the concrete surface 180 has been found to give good results and is also efficient from a cost and complexity perspective.

An example of the angle of departure based positioning technique will now be given, with reference to FIG. 15. Suppose that the inspection tool 100 is initially positioned at a corner of the surface, e.g., at location 1510, with a clear line of sight to two or more rotary laser emitters 160a, 160b, e.g., of the type commonly used at construction sites to establish a horizontal plane reference. Rotary lasers are sometimes also referred to as rotational lasers or laser levels. The inspection tool can be calibrated at the initial location 1510 by detecting the heights h1, h2 of the plurality (two or more) of rotary lasers impinging on the linear photo sensor, and the time each beam hits the sensor. The rotation speeds ω1, ω2 of the rotary lasers can also be determined by measuring the time period inbetween laser beam detections at the same height h1, h2. This calibration results in a mapping between time of detection of a laser beam and angle of departure of the laser beam.

Now, if the inspection tool 100 is moved to another location 1520 on the surface 180, then its position can be determined by measuring the time the laser beams (at the different heights) impinge on the sensor. The time instants each laser beam is detected can be translated into a corresponding angle of departure a1, a2, and the position of the inspection tool 100 on the surface 180 can be determined as the intersection point of two straight lines originating at the laser transmitters 160a, 160b and having the determined angle of departures 160a, 160b. In case there are more than two rotary lasers, then a least squares (LS) fit of position can be made.

This way the inspection tool 100, 800, 820, 900, 910 can be arranged to determine a height of the concrete surface at a given location, and associate this height with a corresponding location on the surface. Thus, a topology 1520, 1530 of the surface can be constructed, e.g., using interpolation between measurement points. The result of this height analysis can be included in an inspection report, and also shown on the display 140.

FIG. 16 shows an example user interface 1600 of the inspection tool. This user interface can be arranged to display the 3D reconstruction 1610 of the surface section where the operator has currently deployed the inspection tool 100, which allows the operator to inspect the surface in detail to determine various properties of the surface. The control system 210, discussed, e.g., in connection to FIG. 2, may advantageously be arranged to compare the plurality of local surface quality values to a pre-configured specification, and to output a validation result 1620 based on the comparison, e.g., via the display 140. Thus, the inspection tool 100 can be used to perform an initial survey of a concrete surface and determine if the surface is ready for a given type of processing. For instance, the inspection tool 100 can be used to survey a concrete surface in order to determine if the surface is ready for processing by a finer grit, or if more processing by a courser grit abrasive tool is necessary due to the presence of scratches and the like. The inspection tool 100 can also be used to validate the result of a concrete processing operation, i.e., to verify that an intended result has been achieved, or if additional processing is required in order to fulfil a requirement specification which can be obtained, e.g., from a remote server 1630.

The control system 210 is optionally also arranged to determine a desired tool selection 1640 based on the determined local surface quality values. The tool selection may be displayed on the display 140.

At least some of the vision-based sensors 110 discussed herein comprise a light source such as one or more LEDs (exemplified in FIG. 5), and/or a projector device arranged to project an image onto the concrete surface 180 (exemplified in FIG. 10). These vision-based sensors also comprise a detector arranged to capture an image of the surface illuminated by the light source. The light source and the detector are spatially separated from each other, meaning that the section of the concrete surface to be inspected is illuminated by the light source from one direction and observed by the detector from another direction. This is an advantage since it allows better detection of unevenness compared to if a laser scanner is used. The angle of separation can be adapted to the specific use case, and some example angles are illustrated in FIG. 5. The structured light set-up also has a spatial separation between light source and detector, as exemplified in FIG. 10. Thus, at least some of the inspection devices described herein comprises a vision-based sensor 110 with one or more light sources and at least one detector, where the light sources are arranged to illuminate a section of the concrete surface from one direction and where the detector is arranged to capture an image or a representation of the section of the concrete surface from another direction compared to the illumination direction. It is appreciated that this can be achieved by a spatial separation of the light source and the detector, optionally in combination with an adjustment of pointing angle of the light source and/or of the detector.

According to some aspects of the inspection devices discussed herein, a light source is arranged to illuminate a section of the concrete surface from a first angle and the detector is arranged to observe the section of the concrete surface from a second angle different from the first angle. The first and second angles can be measured relative to a base plane P of the concrete surface 180, and the magnitude of the difference may be at least five degrees or so, but preferably more as exemplified in FIG. 5 and in FIG. 10.

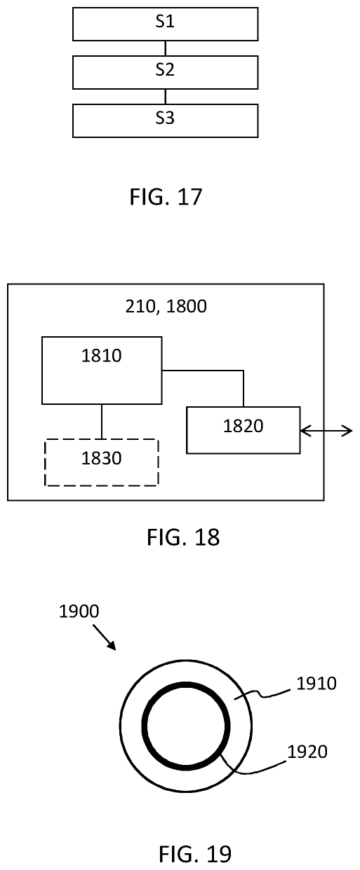

FIG. 17 is a flow chart which illustrates the general concept of concrete surface inspection using the inspection tools 100, 800, 820, 900, 910 discussed herein. There is illustrated a method for inspection of a concrete surface 180, the method comprising, obtaining S1 an inspection tool comprising a vision-based sensor 110 arranged to be directed at a section of the concrete surface 180, where a light shield 115 is configured to enclose the vision-based sensor and the section of the concrete surface 180, guiding means 120 for allowing an operator 170 to move the vision-based sensor 110 over the concrete surface 180, a trigger 130 arranged to receive a command from the operator 170, wherein the vision-based sensor 110 is arranged to capture at least one image of the concrete surface in response to the command, and a control system 210 arranged to analyze the at least one image of the concrete surface in terms of a surface quality of the section of the concrete surface 180, and a display unit 140 arranged to present a result of the surface quality analysis to the operator 170. The method also comprises deploying S2 the inspection tool on a section of a concrete surface 180, and inspecting S3 the section of concrete surface 180 by triggering analysis by the trigger 130.

FIG. 18 schematically illustrates, in terms of a number of functional units, the general components of a control system 1800, such as the control system 210. Processing circuitry 1810 is provided using any combination of one or more of a suitable central processing unit CPU, multiprocessor, microcontroller, digital signal processor DSP, etc., capable of executing software instructions stored in a computer program product, e.g. in the form of a storage medium 1830. The processing circuitry 1810 may further be provided as at least one application specific integrated circuit ASIC, or field programmable gate array FPGA.

Particularly, the processing circuitry 1810 is configured to cause the inspection tool 100 to perform a set of operations, or steps, such as the methods discussed in connection to FIG. 17 and the discussions above. For example, the storage medium 1830 may store the set of operations, and the processing circuitry 1810 may be configured to retrieve the set of operations from the storage medium 1830 to cause the device to perform the set of operations. The set of operations may be provided as a set of executable instructions.

Thus, the processing circuitry 1810 is thereby arranged to execute methods as herein disclosed.

The storage medium 1830 may also comprise persistent storage, which, for example, can be any single one or combination of magnetic memory, optical memory, solid state memory or even remotely mounted memory.

The device 1800 may further comprise an interface 1820 for communications with at least one external device. As such the interface 1820 may comprise one or more transmitters and receivers, comprising analogue and digital components and a suitable number of ports for wireline or wireless communication.

The processing circuitry 1810 controls the general operation of the inspection tool 100, e.g., by sending data and control signals to the interface 1820 and the storage medium 1830, by receiving data and reports from the interface 1820, and by retrieving data and instructions from the storage medium 1830.

FIG. 19 illustrates a computer readable medium 1910 carrying a computer program comprising program code means 1920 for performing the method illustrated in FIG. 17, when said program product is run on a computer. The computer readable medium and the code means may together form a computer program product 1200.

1. An inspection tool for inspection of a concrete surface, the inspection tool comprising
a vision-based sensor arranged to be directed at a section of the concrete surface,
guiding means for allowing an operator to move the vision-based sensor over the concrete surface,
a trigger arranged to receive a command from the operator, wherein the vision-based sensor is arranged to capture at least one image of the concrete surface in response to actuation of the trigger,
a control system arranged to analyze the at least one image of the concrete surface in terms of a surface quality of the section of the concrete surface, and
a data interface and/or a display unit arranged to output and/or to present a result of the surface quality analysis to the operator.

2. The inspection tool according to claim 1, wherein the surface quality analysis comprises presence of scratch marks, cracks in the surface, and/or a level of surface gloss, or wherein the surface quality analysis comprises a suitable grinding tool grit for processing the surface.

3. (canceled)

4. The inspection tool according to claim 1, further comprising a height detection system arranged to detect a height of the inspection tool relative to a reference height, wherein the height detection system comprises an array of photodiodes.

5. The inspection tool according to claim 1, further comprising: a surface cleaning arrangement arranged to remove dust from the surface prior to capture of the at least one image of the concrete surface by the vision-based sensor, or
a lens cleaning arrangement arranged to remove dust from a lens of the vision-based sensor prior to capture of the at least one image of the concrete surface by the vision-based sensor.

6. (canceled)

7. The inspection tool according to claim 5, further comprising a pressurized gas system for dispensing pressurized gas, such as air or carbon dioxide, into an interior of the 3D camera sensor and/or onto the concrete surface and/or onto a lens of the vision-based sensor.

8. The inspection tool according to claim 1, further comprising a positioning system arranged to position the inspection tool on the concrete surface.

9. The inspection tool according to claim 8, wherein the positioning system is based on determination of an angle of departure of a laser beam emitted by a laser transmitter arranged to be supported at a pre-determined distance above a base plane of the concrete surface.

10. The inspection tool according to claim 1, further comprising:
an electrical energy storage device arranged to provide electrical power to the inspection tool,
an internal data storage device configured to store an amount of data associated with the concrete surface, or
an input/output circuit for data, and/or a wireless communications transceiver.

11. (canceled)

12. (canceled)

13. The inspection tool according to claim 1, further comprising a durometer and/or a device arranged to form a scratch in the concrete surface, wherein the durometer and/or the device is arranged to determine a surface hardness level of the concrete surface.

14. The inspection tool according to claim 1, wherein the guiding means comprises:
manual guiding means comprising a handle arranged attached to the vision-based sensor at a distal end of the handle, a trolley supporting the vision-based sensor on a bottom part of the trolley, or a sled arranged to be slidably supported on the concrete surface, or
a remote controlled robot or a robot arranged to move autonomously over the concrete surface, or wherein the inspection tool is integrated in concrete surface processing equipment, such as a floor grinder, a power trowel, or a dust extractor.

15. (canceled)

16. The inspection tool according to claim 1, wherein the vision-based sensor comprises a 3D camera sensor arranged closer than about 30 cm from the concrete surface, and
wherein the 3D camera sensor comprises a plurality of spatially separated light sources and one or more image sensors or a plurality of image sensors arranged for stereoscopic vision.

17. (canceled)

18. (canceled)

19. The inspection tool according to claim 16, wherein the control system is arranged to control the light sources and the at least one image sensor in a shape from shadow, SFS, process to generate a 3D representation of a section of the concrete surface.

20. The inspection tool according to claim 16, wherein the control system is arranged to generate a plurality of 3D representations of the section of the concrete surface by a plurality of image sensors, where the control system is arranged to perform a stereoscopic procedure to determine a 3D representation of the concrete surface comprising depth information.

21. The inspection tool according to claim 16, wherein the control system is arranged to perform a plurality of SFS processes and corresponding stereoscopic process for each elevation angle out of a predetermined plurality of elevation angles.

22. The inspection tool according to claim 1, wherein the vision-based sensor comprises an image sensor with a resolution at or above 13 megapixels or wherein the vision-based sensor comprises a structured light image sensor.

23. (canceled)

24. The inspection tool according to claim 1, wherein a projector component of the structured light image sensor is arranged to operate in a defocused mode of operation.

25. The inspection tool according to claim 1, further comprising an analog or electronic spirit level arranged to indicate an angle of the inspection tool relative to a vertical reference axis.

26. The inspection tool according to claim 1, further comprising at least one laser transmitter arranged to be supported at a pre-determined distance above a base plane of the concrete surface and distanced from the guiding means, or
wherein a light shield is configured to enclose the vision-based sensor and the section of the concrete surface.

27. (canceled)

28. The inspection tool according to claim 1, wherein the vision-based sensor comprises at least one light source such as one or more light emitting diodes, LED, and/or a projector device arranged to project an image onto the concrete surface, and at least one detector arranged to capture an image of the surface, where the light source and the detector are spatially separated from each other, and wherein the light source is arranged to illuminate a section of the concrete surface from a first angle and where the detector is arranged to observe the section of the concrete surface from a second angle different from the first angle.

29. (canceled)

30. A method for inspection of a concrete surface, the method comprising,
obtaining an inspection tool comprising
a vision-based sensor arranged to be directed at a section of the concrete surface,
guiding means for allowing an operator to move the vision-based sensor over the concrete surface,
a trigger arranged to receive a command from the operator, wherein the vision-based sensor is arranged to capture at least one image of the concrete surface in response to the command,
a control system arranged to analyze the at least one image of the concrete surface in terms of a surface quality of the section of the concrete surface, and
a data interface and/or a display unit arranged to output and/or to present a result of the surface quality analysis to the operator,
deploying the inspection tool on a section of a concrete surface, and
inspecting the section of concrete surface by triggering analysis by the trigger.
